(19) United States
(12) Patent Application Publication
(10) Pub. No.: US 2015/0335788 A1
(43) Pub. Date: Nov. 26, 2015

(54) STEM CELL-IMPREGNATED THERAPEUTIC PATCH

(71) Applicant: The Johns Hopkins University, Baltimore, MD (US)

(72) Inventors: Zhiyong Xia, Rockville, MD (US); Chao-Wei Hwang, West Friendship, MD (US); Xiomara Calderon-Colon, Laurel, MD (US); Virginia E. Bogdan, Owings Mills, MD (US); Peter V. Johnston, Baltimore, MD (US)

(21) Appl. No.: 14/639,162

(22) Filed: Mar. 5, 2015

Related U.S. Application Data
(60) Provisional application No. 62/001,745, filed on May 22, 2014.

(51) Int. Cl. A61L 27/38; A61L 27/18; A61L 27/54; A61L 27/56

(52) U.S. Cl. CPC A61L 27/3834; A61L 27/56; A61L 27/18; A61L 27/54; A61L 2430/34; A61L 2400/12; A61L 2300/412

(57) ABSTRACT

A therapeutic patch includes at least one layer of a nanofiber fabric and at least one of a plurality of stem cells or a plurality of stem cell-derived paracrine factors embedded in the nanofiber fabric. The therapeutic patch is produced such that the nanofiber fabric is formed of a nanofiber web.

### CROSS-REFERENCE TO RELATED APPLICATIONS

This application claims the benefit of U.S. Provisional Application No. 62/001,745 filed on May 22, 2014, the entire contents of which are hereby incorporated herein by reference.

### TECHNICAL FIELD

Example embodiments relate generally to therapeutic nanofiber patches, and more particularly to a stem cell-impregnated nanofiber patch that may provide a platform for paracrine factor production and angiogenesis in the body.

### BACKGROUND

Angiogenesis is a crucial step in the healing process and remains a critical challenge in the treatment of injured tissues. Therapeutic angiogenesis also has wide applications beyond wound healing, most notably in treating, for example, coronary artery disease, peripheral vascular disease, and stroke. However, there are currently no approved clinical options for therapeutic angiogenesis because there are at least twenty paracrine factors known to be involved in angiogenesis, but it is unknown how to deliver them in the correct sequence, concentration, and combination to optimize healing. Direct delivery of individual paracrine factors in human clinical trials have had mixed results.

As such, researchers have become increasingly interested in using stem cells to repair tissue injury. Stem cells are capable of producing the entire library of paracrine factors found in the body, including those needed for angiogenesis and tissue regeneration. One technique using stem cells involves directly injecting or infusing stem cells in the target tissue to facilitate regeneration. However, rapid cell dilution, washout, and immune attack limit retention of viable stem cells, and, consequently, diminish the ability of the stem cells to produce sufficient paracrine factors to have desirable clinical effects. Therefore there at least remains a need in the art for a platform from which stem cells can release paracrine factors to promote angiogenesis, while also being shielded from dilution, washout, and immune attack.

### BRIEF SUMMARY

Certain embodiments according to the present invention provide a nanofiber patch suitable for a wide range of therapeutic applications. In accordance with certain embodiments, the therapeutic patch includes at least one layer of a nanofiber fabric and at least one of a plurality of stem cells or a plurality of stem cell-derived paracrine factors embedded in the nanofiber fabric. In certain embodiments, the nanofiber fabric includes a nanofiber web.

In accordance with certain embodiments, a method of treatment for promoting angiogenesis is provided. To promote angiogenesis, the therapeutic patch can be prepared and applied to an object. The therapeutic patch can be prepared to include at least one layer of a nanofiber fabric and at least one of a plurality of stem cells or a plurality of stem cell-derived paracrine factors embedded in the nanofiber fabric. In certain embodiments, the therapeutic patch can be prepared so that the nanofiber fabric includes a nanofiber web.

### BRIEF DESCRIPTION OF THE DRAWING(S)

Example embodiments of the present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout.

FIG. 1 illustrates a top plan view of the therapeutic patch according to an example embodiment.

FIG. 2 illustrates good stem cell viability and clear seeding-dependent cell growth on a therapeutic PLA nanofiber patch after seven days in culture as determined using a Calcein AM-Ethidium homodimer 1 “live-dead” assay in accordance with an example embodiment.

FIG. 3 illustrates that human vascular endothelial cells exposed to therapeutic nanofiber patches containing stem cells spontaneously form tubules in a stem cell dose-dependent manner in accordance with an example embodiment.

FIG. 4 illustrates that freeze-dried therapeutic nanofiber patches induce comparable degrees of human vascular endothelial cell tubule formation as therapeutic nanofiber patches with viable cells.

FIG. 5 illustrates spontaneous tubule formation in human vascular endothelial cells exposed to therapeutic nanofiber patches containing stem cells in accordance with an example embodiment.

FIG. 6A illustrates nanofibers formed using electrospinning conditions in accordance with an example embodiment.

FIG. 6B illustrates nanofibers formed using electrospinning conditions in accordance with an example embodiment.

FIG. 7 illustrates a method of treatment using the therapeutic patch in accordance with an example embodiment.

FIG. 8 illustrates a method of treatment using the therapeutic patch in accordance with an example embodiment.

FIG. 9 illustrates a method of treatment using the therapeutic patch in accordance with an example embodiment.

### DETAILED DESCRIPTION

Some example embodiments now will be described more fully hereinafter with reference to the accompanying drawings, in which some, but not all embodiments of the inventions are shown. Indeed, this invention may be embodied in many different forms and should not be construed as limited to the embodiments set forth herein; rather, these embodiments are provided so that this disclosure will satisfy applicable legal requirements. As used in the specification, and in the appended claims, the singular forms “a”, “an”, “the”, include plural referents unless the context clearly dictates otherwise.

An example embodiment includes a therapeutic patch made of at least one layer of nanofiber fabric with embedded stem cells or stem cell-derived paracrine factors that is applied to an object, which provides a platform from which stem cells can release paracrine factors to promote angiogenesis, while also being shielded from dilution, washout, and immune attack.

The term “tissue”, as used herein, may comprise any component of the body, including, but not limited to, muscle, blood vessels, bone, fat tissue, or skin.

The term “biodegradable”, as used herein, may comprise a tissue-compatible material having the ability to degrade at some time after implantation within the tissue of an animal into nontoxic products which are eliminated from the body or metabolized therein.

The term “nonwoven”, as used herein, may comprise a web having a structure of individual fibers or threads which are interlaid, but not in an identifiable manner as in a knitted or woven fabric. Non-woven fabrics or webs have been formed by many processes such as, for example, meltblowing processes, spunbonding processes, hydroentangling, air-laid, and bonded carded web processes.

The term “nanofiber fabric”, as used herein, may comprise at least one layer of nanofibers processed to form a fabric. The term “nanofiber”, as used herein, may comprise a nonwoven fiber having a thickness from about 1 nanometer to about 2000 nanometers (i.e., 2 micrometers). As used herein, a nanofiber may comprise any biodegradable polymer fiber. The nanofiber fabric layer may comprise a synthetic biocompatible material, a biological material, or any combination thereof. Synthetic biocompatible materials may comprise poly(lactic acid) (“PLA”), poly(L-lactic acid) (“PLLA”), poly(lactic-co-glycolic acid) (“PLGA”), polycaprolactone (“PCL”), poly(ethylene oxide) (“PEO”), poly(ethylene terephthalate) (“PET”), poly(vinyl alcohol) (“PVA”), or any combination thereof. Biological materials may comprise collagen, peptides, proteins, nucleic acids, fatty acids, gelatin, chitosan, hyaluronic acid, or any combination thereof.

The term “nanofiber web”, as used herein, may comprise a nonwoven randomly oriented or aligned collection of nanofibers. These nanofiber webs or mats are typically in the form of a thick and tangled mass defined by an open texture or porosity. For the purposes of this disclosure the terms nanofiber membrane, nanofiber web, nanofiber mat, and nanofiber meshwork are used interchangeably.

The term “pore”, as used herein, may comprise any structure formed by the nonwoven fiber fabric assembly having a maximal pore size. The random arrangement of the nonwoven fibers may create irregular pore structures. Thus, the pores may have irregular shapes and inconsistent sizes generally. As such, the maximal pore size should be understood to correlate to the size of the smallest object that would be retained by or prevented from passing through the pore.

The term “paracrine factor”, as used herein, may comprise one or more members of the entire secretome of a cell. As used herein, paracrine factors may act locally or systemically. Paracrine factors may comprise growth factors, nucleic acids (e.g., micro-RNA), or extracellular vesicles (e.g., exosomes).

The term “stem cell”, as used herein, may comprise hematopoietic or non-hematopoietic cells which exist in almost all tissues and have the capacity of self-renewal and the potential to differentiate into multiple cell types. Tissue injury is associated with the activation of immune/inflammatory cells, not only macrophages and neutrophils but also adaptive immune cells (e.g., CD4⁺ T cells, CD8⁺ T cells, B cells), which are recruited by factors from, for example, apoptotic cells, necrotic cells, damaged microvasculature and stroma. Meanwhile, inflammatory mediators (e.g., TNF-α, IL-1β, free radicals, chemokines, leukotrienes) are often produced by phagocytes in response to damaged cells and spilled cell contents. Thus, these inflammatory molecules and immune cells, together with endothelial cells and fibroblasts, orchestrate changes in the microenvironment that result in the mobilization and differentiation of stem cells into stroma and/or replacement of damaged tissue cells. Once stem cells have entered the microenvironment of injured tissues, for example, many factors (e.g., cytokines such as TNF-α, IL-1, IFN-γ, toxins of infectious agents and hypoxia) can stimulate the release of many factors from the stem cell secretome (e.g., epidermal growth factor (EGF), fibroblast growth factor (FGF), platelet-derived growth factor (PDGF), transforming growth factor-β (TGF-β), vascular endothelial growth factor (VEGF), hepatocyte growth factor (HGF), insulin growth factor-1 (IGF-1), angiopoietin-1 (Ang-1), keratinocyte growth factor (KGF), stromal cell-derived factor-1 (SDF-1)). These growth factors, in turn, promote tissue regeneration and repair.

The term “embedded”, as used herein, may generally refer to the placement and capture of cells within nanofiber fabric either secured within the nanofiber fabric or on the surface of the nanofiber fabric. For the purposes of this disclosure, the terms embedded, caged, anchored, or otherwise retained may be used interchangeably.

The term “medical device”, as used herein, may comprise any medical instrument to be used in conjunction with a therapeutic patch. Medical devices may comprise, for example, stents, pacemakers, vascular grafts, implantable cardioverter-defibrillators, pacemaker leads, implantable cardioverter-defibrillator leads, biventricular implantable cardioverter-defibrillator leads, artificial hearts, artificial valves, ventricular assist devices, balloon pumps, catheters, central venous lines, orthopedic implants, wound healing packing, or wound healing gauze.

I. Therapeutic Patch

In some example embodiments, a therapeutic patch suitable for a wide variety of end-uses is provided. Therapeutic patches, according to some example embodiments, may include many desirable features for delivering stem cell-based therapies, such as tailored local paracrine factor release to promote angiogenesis, prevention of cell dilution and washout, and protection from immune attack, to name just a few. In general, therapeutic patches according to some example embodiments may comprise at least one layer of a nanofiber fabric and at least one of a plurality of stem cells or a plurality of stem cell-derived paracrine factors embedded, caged, anchored, or otherwise retained in the nanofiber fabric. According to certain embodiments, the nanofiber fabric may comprise a nanofiber web.

In such embodiments, for instance, the nanofiber web may comprise a nonwoven randomly oriented or aligned collection of nanofibers. In some embodiments, for example, the nanofiber web may be in the form of a thick and tangled mass defined by an open texture or porosity. According to certain embodiments, for instance, the nanofiber fabric may be formed using an electrospinning production process. In such embodiments, the morphologies of the nanofiber fabric may be arbitrarily controlled using different electrospinning settings. In other embodiments, for example, the nanofiber fabric may be formed using a solid state polymer multilayer extrusion process.

In accordance with an example embodiment, the nanofiber fabric may comprise a biodegradable material. In some embodiments, for example, the nanofiber fabric may comprise biodegradable polymer-based fibers. In such embodiments, for instance, the biodegradable polymer-based fibers may decompose and become part of the body over time.

According to certain embodiments, the nanofiber fabric may comprise at least one of a synthetic biocompatible material, a biological material, or any combination thereof. In certain embodiments, the synthetic biocompatible material may comprise at least one of poly(lactic acid) (“PLA”), poly(L-lactic acid) (“PLLA”), poly(lactic-co-glycolic acid) (“PLGA”), polycaprolactone (“PCL”), poly(ethylene oxide) (“PEO”), poly(ethylene terephthalate) (“PET”), poly(vinyl alcohol) (“PVA”), or any combination thereof. In certain embodiments, for example, the synthetic biocompatible material may comprise PLA. In such embodiments, for instance, the synthetic biocompatible material may comprise non-medical grade PLA. In other embodiments, for example, the synthetic biocompatible material may comprise PLGA. In such embodiments, for instance, the synthetic biocompatible material may comprise medical grade PLGA. In accordance with certain embodiments, the biological material may comprise at least one of collagen, peptides, proteins, nucleic acids, fatty acids, gelatin, chitosan, hyaluronic acid, or any combination thereof. In some embodiments, for example, the biological material may comprise collagen.

In accordance with an example embodiment, for instance, the nanofiber fabric may comprise a fiber diameter from about 80 nanometers to about 2 micrometers (i.e., 2000 nanometers). In further embodiments, for example, the nanofiber fabric may comprise a fiber diameter from about 100 nanometers to about 1 micrometer (i.e., 1000 nanometers). In other embodiments, for instance, the nanofiber fabric may comprise a fiber diameter from about 250 nanometers to about 750 nanometers. In some embodiments, for example, the nanofiber fabric may comprise a fiber diameter of about 500 nanometers. As such, in certain embodiments, the nanofiber fabric may comprise a fiber diameter from at least about any of the following: 80, 100, 250, 300, 400, and 500 nm and/or at most about 2000, 1000, 750, 700, 600, and 500 nm (e.g., about 100-700 nm, about 400-600 nm, etc.).

In accordance with an example embodiment, for instance, the nanofiber fabric may define a plurality of pores. For example, the pores may be configured to have a maximal pore size less than the embedded cell diameter. Thus, the pores in the nanofiber fabric layer(s) may act as a “cage” for the embedded cells, thereby preventing cell dilution and washout. In some embodiments, for instance, the pores may be further configured to have a maximal pore size less than other cell diameters. In this regard, the maximal pore size may be less than any immune cell diameter, thereby protecting the embedded cells from immune attack. In certain embodiments, for example, the pores may be further configured to have a maximal pore size greater than any paracrine factors, nutrients required by the embedded cells, and any wastes or cellular products produced by the embedded cells. In this regard, the maximal pore size may accommodate the needs of the cells embedded therein. In this regard, certain embodiments may comprise protection against immune attack, dilution, and cell washout while further providing a beneficial environment for the embedded cell health.

In accordance with an example embodiment, for instance, the nanofiber fabric may comprise a pore size from about 100 nm to about 7 μm. In further embodiments, for example, the nanofiber fabric may comprise a pore size from about 200 nm to about 6 μm. In other embodiments, for instance, the nanofiber fabric may comprise a pore size from about 300 nm to about 5 μm. In certain embodiments, for example, the nanofiber fabric may comprise a pore size from about 500 nm to about 1 μm. As such, in certain embodiments, the nanofiber fabric may comprise a pore size from at least about any of the following: 100, 200, 300, 400, and 500 nm and/or at most about 7, 6, 5, 3, and 1 μm (e.g., about 100 nm-1 μm, about 500 nm-1 μm, etc.).

In accordance with an example embodiment, the nanofiber fabric in the therapeutic patch may act as a scaffold for vascular tubules to form, a prerequisite for angiogenesis. In such an embodiment, after stem cells are embedded in the nanofiber fabric to form the therapeutic patch, other cells (e.g., human vascular endothelial cells) may attach to the nanofibers, elongate, and proliferate. Additional cells (e.g., human vascular endothelial cells) may continue attaching to the cells already anchored to the nanofibers to form cell channels attached to the nanofibers. Because the nanofibers have a high surface to volume ratio, the cell channels may exhibit a sufficient size and shape to form vascular tubules as the biodegradable nanofibers decompose and become part of the body. As such, the vascular tubules may initiate angiogenesis.

In accordance with an example embodiment, the therapeutic patch may comprise stem cell seeding at different densities. In such an embodiment, for instance, the therapeutic patch may comprise stem cell seeding at a density of at most 10⁶ cells/cm². At higher cell seeding densities, overcrowding may limit stem cell viability and vascular tubule formation. In other embodiments, for example, the therapeutic patch may comprise stem cell seeding at a density from about 10⁵ cells/cm² to about 10⁶ cells/cm². In further embodiments, for instance, the therapeutic patch may comprise stem cell seeding at a density from about 2×10⁵ cells/cm² to about 6×10⁵ cells/cm². In certain embodiments, for example, the therapeutic patch may comprise stem cell seeding at a density of 4×10⁵ cells/cm². As such, in certain embodiments, the therapeutic patch may comprise stem cell seeding at a density from at least about any of the following: 10⁵, 2×10⁵, 3×10⁵, and 4×10⁵ cells/cm² and/or at most about 10⁶, 6×10⁵, 5×10⁵, and 4×10⁵ cells/cm² (e.g., about 3×10⁵-6×10⁵ cells/cm², about 2×10⁵-4×10⁵ cells/cm², etc.).

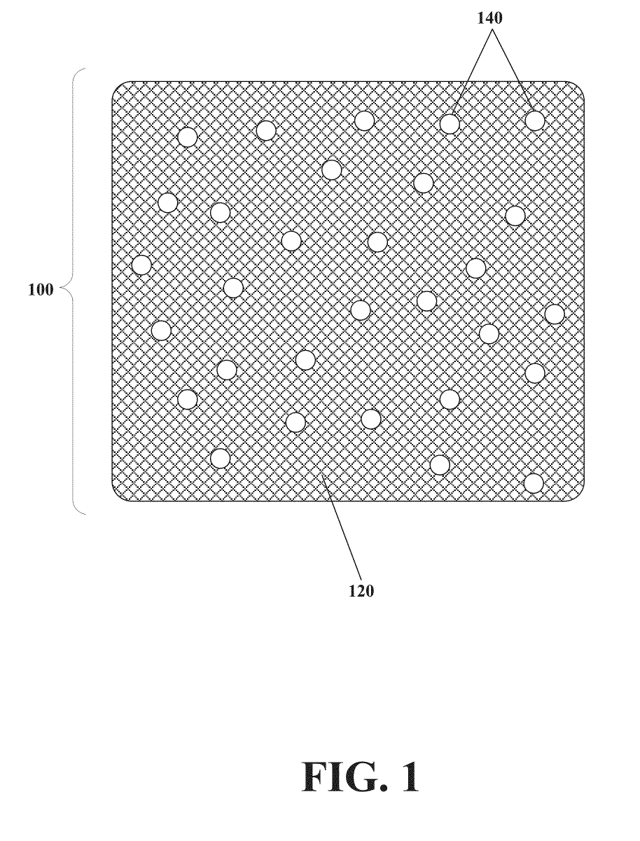

For example, FIG. 1 illustrates a top plan view of the therapeutic patch according to an example embodiment. As shown in FIG. 1, the therapeutic patch 100 illustrated in FIG. 1 includes nanofiber fabric 120 and a plurality of stem cells 140 embedded in the nanofiber fabric 120.

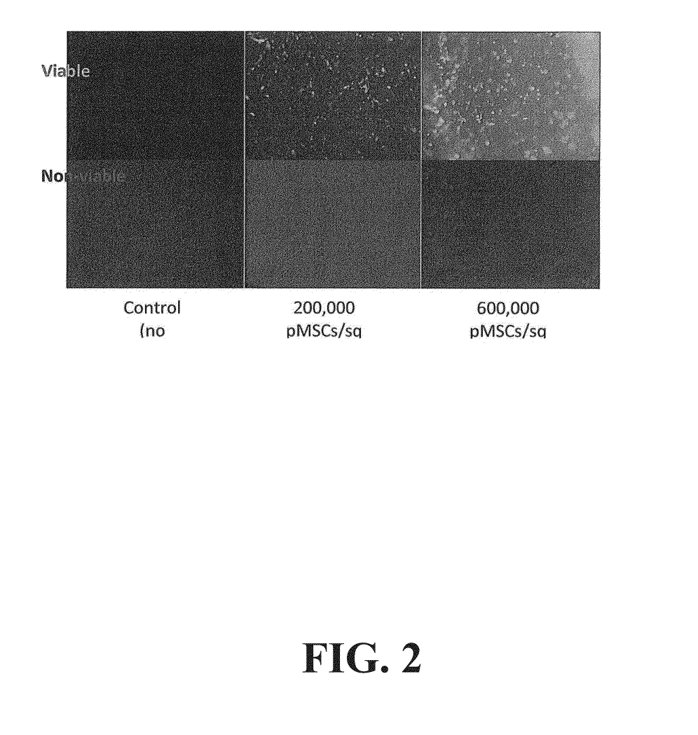

FIG. 2, for example, illustrates good stem cell viability and clear seeding-dependent cell growth on a therapeutic PLA nanofiber patch after seven days in culture as determined using a Calcein AM-Ethidium homodimer 1 “live-dead” assay. As shown in FIG. 2, viable stem cells (top row) cultured in a therapeutic PLA nanofiber patch far outnumber non-viable stem cells (bottom row) after seven days in culture.

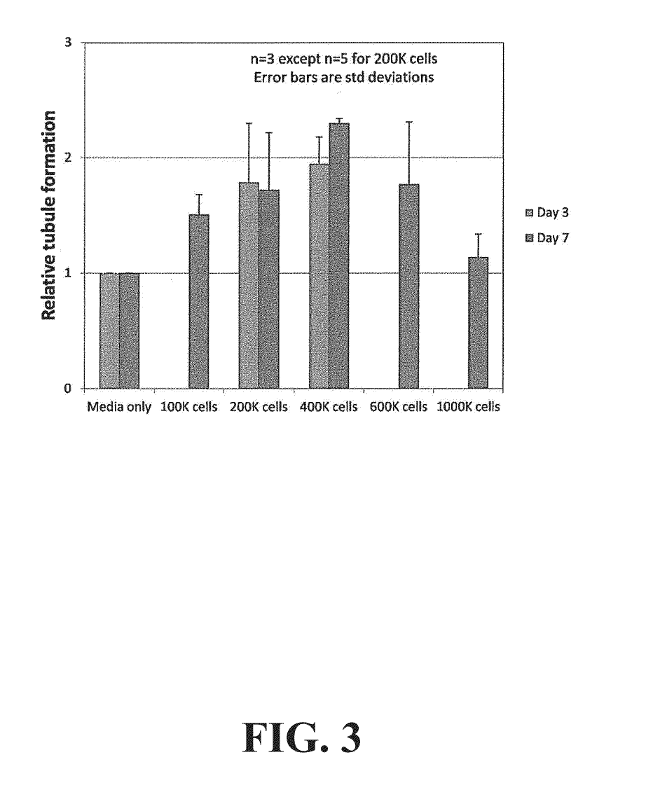

FIG. 3, for example, illustrates that human vascular endothelial cells exposed to therapeutic nanofiber patches containing stem cells spontaneously form tubules in a stem cell dose-dependent manner. As shown in FIG. 3, vascular tubule formation depends on therapeutic patch cell seeding density, with peak formation at a density of 4×10⁵ cells/cm² when tubule formation is in excess of two-fold that of the control. Furthermore, FIG. 3 illustrates that even after adsorbent paracrine factors are removed from therapeutic patches, the cell-seeded therapeutic patches can successfully regenerate enough paracrine factors both at Day 3 and Day 7 to stimulate human vascular endothelial cells to spontaneously form vascular tubules.

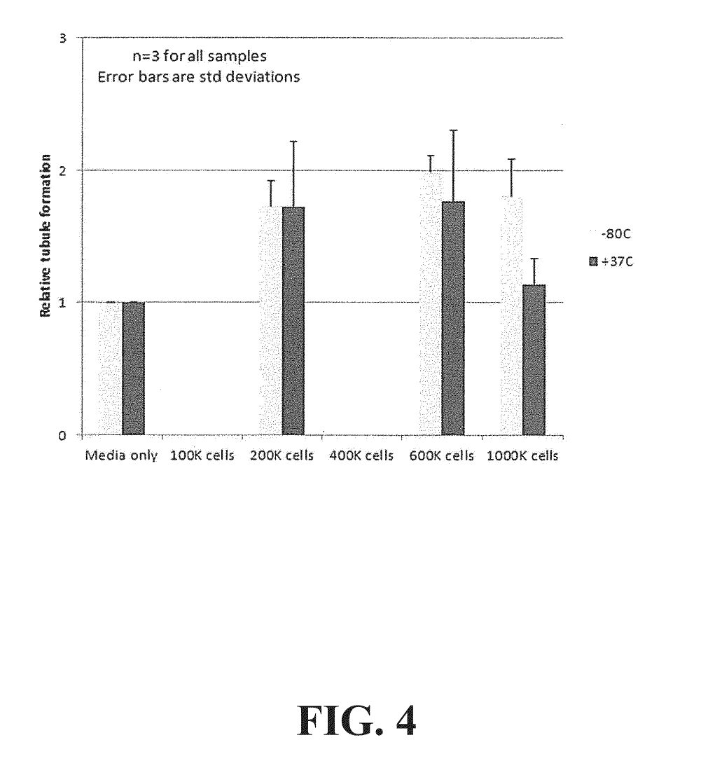

FIG. 4, for example, illustrates that freeze-dried therapeutic nanofiber patches induce comparable degrees of human vascular endothelial cell tubule formation as therapeutic nanofiber patches with viable cells. As shown in FIG. 4, therapeutic patches containing only stem cell-derived paracrine factors (i.e., the freeze-dried therapeutic patches) successfully induce human vascular endothelial cells to form vascular tubules at comparable levels as those therapeutic patches containing viable stem cells.

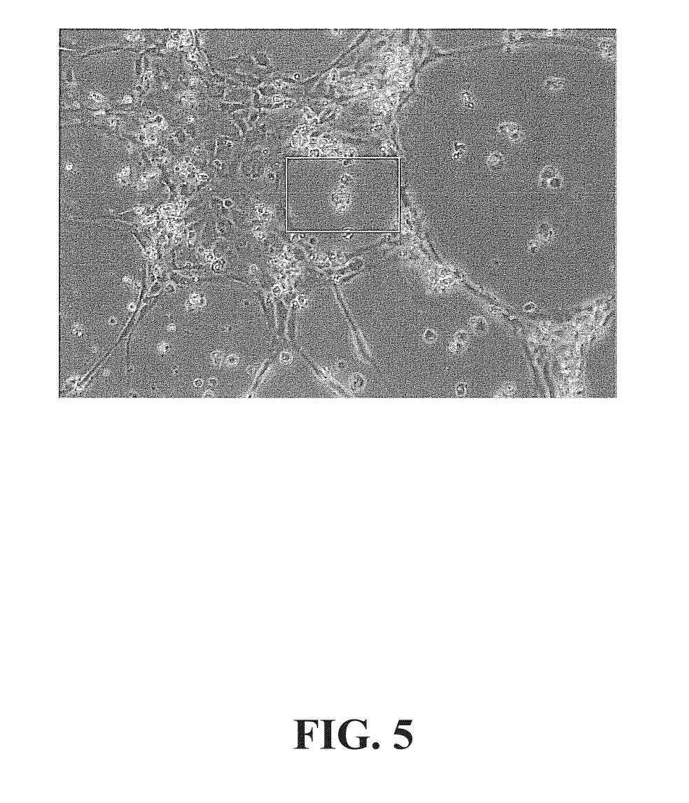

FIG. 5, for example, illustrates spontaneous tubule formation in human vascular endothelial cells exposed to therapeutic nanofiber patches containing stem cells. As shown in FIG. 5, human vascular endothelial cells exposed to therapeutic patches containing stem cells will aggregate and eventually begin to form vascular tubules.

II. Method of Treatment

In another aspect, some example embodiments provide a method of treatment for promoting angiogenesis. In general, the method of treatment, according to some example embodiments, may include preparing a therapeutic patch and applying the therapeutic patch to an object. In certain embodiments, the therapeutic patch may comprise at least one layer of a nanofiber fabric and at least one of a plurality of stem cells or a plurality of stem cell-derived paracrine factors embedded, caged, anchored, or otherwise retained in the nanofiber fabric. According to certain embodiments, the nanofiber fabric may comprise a nanofiber web.

In accordance with an example embodiment, applying a therapeutic patch to an object may comprise applying the therapeutic patch to at least one of a tissue, an organ or portion of an organ, a blood vessel, a muscle, a bone, a joint, a bypass graft, a medical device, or any combination thereof. According to certain embodiments, for instance, applying a therapeutic patch to an object may comprise applying the therapeutic patch to at least one of a tissue, an organ or portion of an organ, a blood vessel, a muscle, a bone, or a joint. In such embodiments, the therapeutic patch may comprise stem cells and/or stem-cell derived paracrine factors. In some embodiments, for example, the therapeutic patch may be placed within or around at least one of a tissue, an organ or portion of an organ, a blood vessel, a muscle, a bone, or a joint. In such embodiments, for instance, the therapeutic patch may accelerate post-operative healing.

According to certain embodiments, applying a therapeutic patch to an object may comprise applying the therapeutic patch to a medical device. In such embodiments, for example, applying a therapeutic patch to an object may comprise applying the therapeutic patch to a stent, a pacemaker, an implantable cardioverter-defibrillator, a pacemaker lead, an implantable cardioverter-defibrillator lead, an artificial heart, an artificial valve, a ventricular assist device, a balloon pump, a catheter, a central venous line, an orthopedic implant, wound healing packing, or wound healing gauze. In certain embodiments, for instance, applying a therapeutic patch to an object may comprise applying the therapeutic patch to a stent. In certain embodiments, for example, the therapeutic patch may be directly sewn and/or adhered onto the surface of the stent. In other embodiments, for instance, the therapeutic patch may be immobilized between at least two concentrically oriented stents and deployed as one unit. In some embodiments, for example, the therapeutic patch may cover the entire stent. In further embodiments, for instance, the therapeutic patch may cover only one or more parts of the stent. In certain embodiments, the therapeutic patch may comprise stem cells and/or stem cell-derived paracrine factors. According to certain embodiments, for example, the therapeutic patch may be applied to a coronary or endovascular stent to promote myocardial or tissue regeneration and angiogenesis.

In accordance with an example embodiment, preparing a therapeutic patch may comprise preparing a single-use therapeutic patch including stem cell-derived paracrine factors. In such an embodiment, preparing the single-use therapeutic patch may comprise at least one of directly absorbing conditioned media into the therapeutic patch, immersing the therapeutic patch in a homogenized cell solution, or lysing stem cells in the therapeutic patch. According to certain embodiments, for instance, the single-use therapeutic patch may be portable and removed and/or replaced after a period of time as necessary.

In accordance with an example embodiment, preparing a therapeutic patch may comprise preparing a reusable therapeutic patch including stem cells. In such an embodiment, preparing the reusable therapeutic patch may comprise seeding stem cells into the therapeutic patch and incubating the stem cells in the therapeutic patch. In certain embodiments, for example, the stem cells within the therapeutic patch may sense the evolving needs of the healing tissue and adjust paracrine factor production accordingly.

In accordance with an example embodiment, the nanofiber fabric may comprise a biodegradable material. In some embodiments, for instance, the nanofiber fabric may comprise biodegradable polymer-based fibers. In such embodiments, for example, the biodegradable polymer-based fibers may break down and become part of the body over time.

According to certain embodiments, the nanofiber fabric may comprise at least one of a synthetic biocompatible material, a biological material, or any combination thereof. In certain embodiments, the synthetic biocompatible material may comprise at least one of poly(lactic acid) (“PLA”), poly(L-lactic acid) (“PLLA”), poly(lactic-co-glycolic acid) (“PLGA”), polycaprolactone (“PCL”), poly(ethylene oxide) (“PEO”), poly(ethylene terephthalate) (“PET”), poly(vinyl alcohol) (“PVA”), or any combination thereof. In certain embodiments, for instance, the synthetic biocompatible material may comprise PLA. In such embodiments, for example, the synthetic biocompatible material may comprise non-medical grade PLA. In other embodiments, for instance, the synthetic biocompatible material may comprise PLGA. In such embodiments, for example, the synthetic biocompatible material may comprise medical grade PLGA. In some embodiments, the biological material may comprise at least one of collagen, peptides, proteins, nucleic acids, fatty acids, gelatin, chitosan, hyaluronic acid, or any combination thereof. In certain embodiments, for instance, the biological material may comprise collagen.

In accordance with an example embodiment, for example, the nanofiber fabric may comprise a fiber diameter from about 80 nanometers to about 2 micrometers (i.e., 2000 nanometers). In further embodiments, for instance, the nanofiber fabric may comprise a fiber diameter from about 100 nanometers to about 1 micrometer (i.e., 1000 nanometers). In other embodiments, for example, the nanofiber fabric may comprise a fiber diameter from about 250 nanometers to about 750 nanometers. In some embodiments, for instance, the nanofiber fabric may comprise a fiber diameter of about 500 nanometers. As such, in certain embodiments, the nanofiber fabric may comprise a fiber diameter from at least about any of the following: 80, 100, 250, 300, 400, and 500 nm and/or at most about 2000, 1000, 750, 700, 600, and 500 nm (e.g., about 100-700 nm, about 400-600 nm, etc.).

In accordance with an example embodiment, the nanofiber fabric may comprise a nanofiber web. In such an embodiment, the nanofiber web may comprise a nonwoven randomly oriented or aligned collection of nanofibers. In some embodiments, for example, the nanofiber web may be in the form of a thick and tangled mass defined by an open texture or porosity. According to certain embodiments, for instance, the nanofiber fabric may be formed using an electrospinning production process. In such embodiments, the morphologies of the nanofiber fabric may be arbitrarily controlled using different electrospinning settings. In other embodiments, for example, the nanofiber fabric may be formed using a solid state polymer multilayer extrusion process.

In accordance with an example embodiment, the therapeutic patch may comprise stem cell seeding at different densities. In such an embodiment, for instance, the therapeutic patch may comprise stem cell seeding at a density of at most 10⁶ cells/cm². In other embodiments, for example, the therapeutic patch may comprise stem cell seeding at a density from about 10⁵ cells/cm² to about 10⁶ cells/cm². In further embodiments, for instance, the therapeutic patch may comprise stem cell seeding at a density from about 2×10⁵ cells/cm² to about 6×10⁵ cells/cm². In certain embodiments, for example, the therapeutic patch may comprise stem cell seeding at a density of 4×10⁵ cells/cm². As such, in certain embodiments, the therapeutic patch may comprise stem cell seeding at a density from at least about any of the following: 10⁵, 2×10⁵, 3×10⁵, and 4×10⁵ cells/cm² and/or at most about 10⁶, 6×10⁵, 5×10⁵, and 4×10⁵ cells/cm² (e.g., about 3×10⁵-6×10⁵ cells/cm², about 2×10⁵-4×10⁵ cells/cm², etc.).

In accordance with an example embodiment, the nanofiber fabric in the therapeutic patch may act as a scaffold for vascular tubules to form, a prerequisite for angiogenesis. In such an embodiment, after stem cells are embedded in the nanofiber fabric to form the therapeutic patch, other cells (e.g., human vascular endothelial cells) may attach to the nanofibers, elongate, and proliferate. Additional cells (e.g., human vascular endothelial cells) may continue attaching to the cells already anchored to the nanofibers to form cell channels attached to the nanofibers. Because the nanofibers have a high surface to volume ratio, the cell channels may exhibit a sufficient size and shape to form vascular tubules as the biodegradable nanofibers decompose and become part of the body. As such, the vascular tubules may begin angiogenesis.

In accordance with an example embodiment, for instance, the nanofiber fabric may define a plurality of pores. For example, the pores may be configured to have a maximal pore size less than the embedded cell diameter. Thus, the pores in the nanofiber fabric layer(s) may act as a “cage” for the embedded cells, thereby preventing cell dilution and washout. In some embodiments, for instance, the pores may be further configured to have a maximal pore size less than other cell diameters. In this regard, the maximal pore size may be less than any immune cell diameter, thereby protecting the embedded cells from immune attack. In certain embodiments, for example, the pores may be further configured to have a maximal pore size greater than any paracrine factors, nutrients required by the embedded cells, and any wastes or cellular products produced by the embedded cells. In this regard, the maximal pore size may accommodate the needs of the cells embedded therein. In this regard, certain embodiments may comprise protection against immune attack, dilution, and cell washout while further providing a beneficial environment for the embedded cell health.

In accordance with an example embodiment, for instance, the nanofiber fabric may comprise a pore size from about 100 nm to about 7 μm. In further embodiments, for example, the nanofiber fabric may comprise a pore size from about 200 nm to about 6 μm. In other embodiments, for instance, the nanofiber fabric may comprise a pore size from about 300 nm to about 5 μm. In certain embodiments, for example, the nanofiber fabric may comprise a pore size from about 500 nm to about 1 μm. As such, in certain embodiments, the nanofiber fabric may comprise a pore size from at least about any of the following: 100, 200, 300, 400, and 500 nm and/or at most about 7, 6, 5, 3, and 1 μm (e.g., about 100 nm-1 μm, about 500 nm-1 μm, etc.).

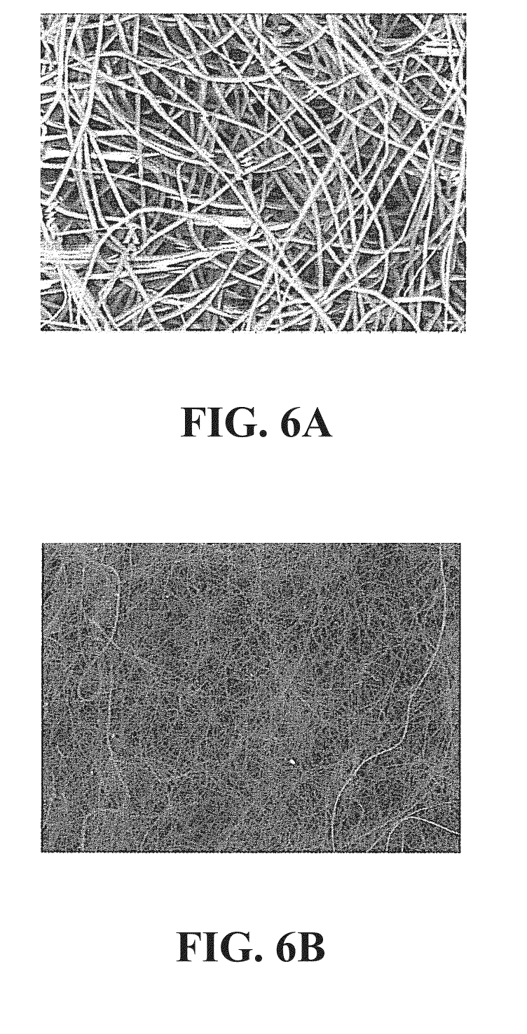

For example, FIGS. 6A and 6B illustrate nanofibers formed using electrospinning conditions according to an example embodiment. As shown in FIGS. 6A and 6B, electrospinning PLA nanofibers from different solutions and with different voltages may significantly affect the nanofiber web structure of the therapeutic patch. Specifically, FIG. 6A illustrates a nanofiber web formed through electrospinning with a lower voltage than FIG. 6B. Additionally, FIG. 6A illustrates a nanofiber web formed using a 100% dichloroethane solution, while FIG. 6B illustrates a nanofiber web formed using a solution of dichloroethane mixed with methanol.

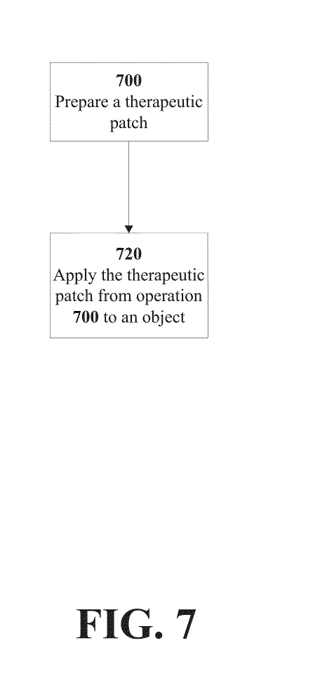

FIG. 7, for example, illustrates a method of treatment using the therapeutic patch according to an example embodiment. As shown in FIG. 7, the method comprises preparing a therapeutic patch in operation 700. The method further comprises applying the therapeutic patch from operation 700 to an object in operation 720.

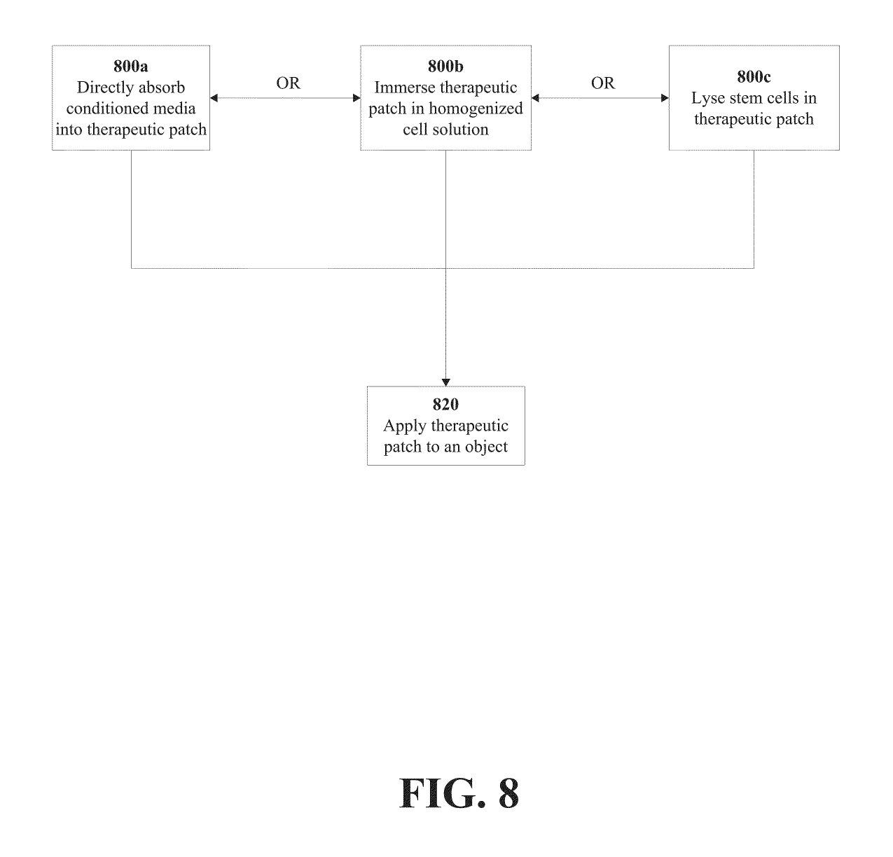

FIG. 8, for example, illustrates a method of treatment using a single-use therapeutic patch according to an example embodiment. As shown in FIG. 8, the method comprises directly absorbing conditioned media into the therapeutic patch in operation 800a, immersing the therapeutic patch in a homogenized cell solution in operation 800b, or lysing stem cells in the therapeutic patch in operation 800c. Each of the alternative operations 800a-800c comprises the preparation step of the method as recited in operation 700 in FIG. 7. The method further comprises applying the therapeutic patch to an object in operation 820.

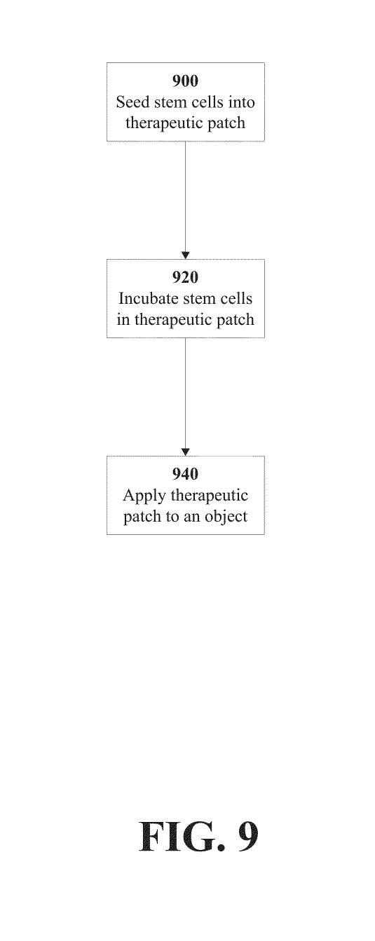

FIG. 9, for example, illustrates a method of treatment using a reusable therapeutic patch according to an example embodiment. As shown in FIG. 9, the method comprises seeding stem cells into the therapeutic patch in operation 900 and incubating the stem cells in the therapeutic patch in operation 920. The method further comprises applying the therapeutic patch to an object in operation 940.

### Examples

The present disclosure is further illustrated by the following examples, which in no way should be construed as being limiting. That is, the specific features described in the following examples are merely illustrative, and not limiting.

Therapeutic Patch Production

Nanofibers are spun via an electrospinning process. In the electrospinning process, 15 wt. % PLA is dissolved in organic solvents for establishing the right solution with proper surface tension. The solvent may be 100% dichloroethane so that the total polymer solution volume is 2 mL or a solution of dichloroethane mixed with methanol in a ratio of 75:25 dichloroethane:methanol. The electrospinning voltage is controlled from 5-16 kV. During the electrospinning process, a syringe pump is used to eject dissolved polymer through a 22 gauge needle at a flow rate of 2 mL/h to a collecting target at a distance of 15 cm.

Cell Incorporation into Therapeutic Patch

The therapeutic nanofiber patches (approximately 10×10×0.5 mm³) are sterilized with 70% EtOH for 18 hours, washed in PBS buffer, and immersed in 20% FBS Alpha MEM media. Stem cells (e.g., porcine mesenchymal stem cells) are then seeded on the patch at different densities of up to 10⁶ cells/cm². The cells are then grown in a humidity and CO₂ controlled incubator for several days.

Cell-Derived Paracrine Factor Incorporation into Therapeutic Patch

Stem cells (e.g., porcine mesenchymal stem cells) are cultured in PLA nanofiber patches for a 7 day period, followed by a freeze-drying process at −80 C. Freeze-drying the stem cells lyses the cells in situ and causes the cells to release their paracrine factor contents directly into the therapeutic patch. These therapeutic patches successfully induce human vascular endothelial cells to form vascular tubules at levels comparable to therapeutic patches containing viable stem cells.

### Exemplary Embodiments

Having described various aspects and embodiments of the invention herein, further specific embodiments of the invention include those set forth in the following paragraphs.

In some example embodiments, therapeutic patches suitable for a wide range of therapeutic uses are provided. In one aspect, a therapeutic patch according to certain exemplary embodiments comprises at least one layer of a nanofiber fabric and at least one of a plurality of stem cells or a plurality of stem cell-derived paracrine factors embedded, caged, anchored, or otherwise retained in the nanofiber fabric. According to certain embodiments, the nanofiber fabric comprises a nanofiber web.

In accordance with an example embodiment, the nanofiber fabric comprises a fiber diameter from about 80 nanometers to about 2 micrometers. In further embodiments, the nanofiber fabric comprises a fiber diameter from about 100 nanometers to about 1 micrometer.

In accordance with an example embodiment, the nanofiber fabric comprises a plurality of pores. In such embodiments, the pores are configured to have a maximal pore size less than the embedded cell diameter. Thus, the pores in the nanofiber fabric layer(s) act as a “cage” for the embedded cells, thereby preventing cell dilution and washout. In some embodiments, the pores are further configured to have a maximal pore size less than other cell diameters. In this regard, the maximal pore size is less than any immune cell diameter, thereby protecting the embedded cells from immune attack. In certain embodiments, the pores are further configured to have a maximal pore size greater than any paracrine factors, nutrients required by the embedded cells, and any wastes or cellular products produced by the embedded cells. In this regard, the maximal pore size accommodates the needs of the cells embedded therein. In this regard, certain embodiments comprise protection against immune attack, dilution, and cell washout while further providing a beneficial environment for the embedded cell health.

In accordance with an example embodiment, the nanofiber fabric comprises a biodegradable material. According to certain embodiments, the nanofiber fabric comprises at least one of a synthetic biocompatible material, a biological material, or any combination thereof. In certain embodiments, the synthetic biocompatible material comprises at least one of poly(lactic acid), poly(L-lactic acid), poly(lactic-co-glycolic acid), polycaprolactone, poly(ethylene oxide), poly(ethylene terephthalate), poly(vinyl alcohol), or any combination thereof. In accordance with certain embodiments, the biological material comprises at least one of collagen, peptides, proteins, nucleic acids, fatty acids, gelatin, chitosan, hyaluronic acid, or any combination thereof.

In another aspect, a method of treatment for promoting angiogenesis is provided. In general, the method of treatment, according to certain example embodiments, includes preparing a therapeutic patch and applying the therapeutic patch to an object. In certain embodiments, the therapeutic patch comprises at least one layer of a nanofiber fabric and at least one of a plurality of stem cells or a plurality of stem cell-derived paracrine factors embedded, caged, anchored, or otherwise retained in the nanofiber fabric. According to certain embodiments, the nanofiber fabric comprises a nanofiber web.

In accordance with an example embodiment, the nanofiber fabric comprises a fiber diameter from about 80 nanometers to about 2 micrometers. In further embodiments, the nanofiber fabric comprises a fiber diameter from about 100 nanometers to about 1 micrometer.

In accordance with an example embodiment, the nanofiber fabric comprises a plurality of pores. In such embodiments, the pores are configured to have a maximal pore size less than the embedded cell diameter. Thus, the pores in the nanofiber fabric layer(s) act as a “cage” for the embedded cells, thereby preventing cell dilution and washout. In some embodiments, the pores are further configured to have a maximal pore size less than other cell diameters. In this regard, the maximal pore size is less than any immune cell diameter, thereby protecting the embedded cells from immune attack. In certain embodiments, the pores are further configured to have a maximal pore size greater than any paracrine factors, nutrients required by the embedded cells, and any wastes or cellular products produced by the embedded cells. In this regard, the maximal pore size accommodates the needs of the cells embedded therein. In this regard, certain embodiments comprise protection against immune attack, dilution, and cell washout while further providing a beneficial environment for the embedded cell health.

In accordance with an example embodiment, the nanofiber fabric comprises a biodegradable material. According to certain embodiments, the nanofiber fabric comprises at least one of a synthetic biocompatible material, a biological material, or any combination thereof. In certain embodiments, the synthetic biocompatible material comprises at least one of poly(lactic acid), poly(L-lactic acid), poly(lactic-co-glycolic acid), polycaprolactone, poly(ethylene oxide), poly(ethylene terephthalate), poly(vinyl alcohol), or any combination thereof. In accordance with certain embodiments, the biological material comprises at least one of collagen, peptides, proteins, nucleic acids, fatty acids, gelatin, chitosan, hyaluronic acid, or any combination thereof.

In accordance with an example embodiment, applying a therapeutic patch to an object comprises applying the therapeutic patch to at least one of a tissue, an organ or portion of an organ, a blood vessel, a muscle, a bone, a joint, a bypass graft, a medical device, or any combination thereof. In certain embodiments, applying a therapeutic patch to an object comprises applying the therapeutic patch to a stent, a pacemaker, an implantable cardioverter-defibrillator, a pacemaker lead, an implantable cardioverter-defibrillator lead, an artificial heart, an artificial valve, a ventricular assist device, a balloon pump, a catheter, a central venous line, an orthopedic implant, wound healing packing, or wound healing gauze.

In accordance with an example embodiment, preparing a therapeutic patch comprises preparing a single-use therapeutic patch including stem cell-derived paracrine factors. In such embodiments, preparing the single-use therapeutic patch comprises at least one of directly absorbing conditioned media into the therapeutic patch, immersing the therapeutic patch in a homogenized cell solution, or lysing stem cells in the therapeutic patch. In other embodiments, preparing a therapeutic patch comprises preparing a reusable therapeutic patch including stem cells. In such embodiments, preparing the reusable therapeutic patch comprises seeding stem cells into the therapeutic patch and incubating the stem cells in the therapeutic patch.

These and other modifications and variations to the present invention may be practiced by those of ordinary skill in the art without departing from the spirit and scope of the present invention, which is more particularly set forth in the appended claims. In addition, it should be understood that aspects of the various embodiments may be interchanged in whole or in part. Furthermore, those of ordinary skill in the art will appreciate that the foregoing description is by way of example only, and it is not intended to limit the invention as further described in such appended claims. Therefore, the spirit and scope of the appended claims should not be limited to the exemplary description of the versions contained herein.

What is claimed is:

1. A therapeutic patch, comprising:
at least one layer of a nanofiber fabric; and
at least one of a plurality of stem cells and a plurality of stem cell-derived paracrine factors embedded in the nanofiber fabric,
wherein the nanofiber fabric comprises a nanofiber web.

2. The therapeutic patch according to claim 1, wherein the nanofiber fabric comprises a nonwoven nanofiber fabric having a fiber diameter from about 80 nm to about 2 μm.

3. The therapeutic patch according to claim 1, wherein the nanofiber fabric comprises a plurality of pores, said plurality of pores comprising a maximal pore size.

4. The therapeutic patch according to claim 3, wherein the embedded stem cells comprise an embedded stem cell diameter, and
wherein the maximal pore size is less than the embedded stem cell diameter, the maximal pore size is less than other cell diameters, and the maximal pore size is greater than embedded paracrine factor, cell product, nutrient, or waste diameters.

5. The therapeutic patch according to claim 1, wherein the nanofiber fabric comprises a biodegradable polymer.

6. The therapeutic patch according to claim 4, wherein the nanofiber fabric comprises at least one of a synthetic biocompatible material, a biological material, and any combination thereof.

7. The therapeutic patch according to claim 5, wherein the nanofiber fabric comprises at least one of poly(L-lactic acid), poly(lactic-co-glycolic acid), polycaprolactone, polyethylene oxide, poly(ethylene terephthalate), poly(vinyl alcohol), collagen, and any combination thereof.

8. A method of treatment, comprising:
preparing a therapeutic patch comprising:
at least one layer of a nanofiber fabric; and
at least one of a plurality of stem cells or a plurality of stem cell-derived paracrine factors embedded in the nanofiber fabric; and
applying the therapeutic patch to an object,
wherein the nanofiber fabric comprises a nanofiber web.

9. The method of treatment according to claim 8, wherein the nanofiber fabric comprises a nonwoven nanofiber fabric having a fiber diameter from about 80 nm to about 2 μm.

10. The method of treatment according to claim 8, wherein the nanofiber fabric comprises a plurality of pores, said plurality of pores comprising a maximal pore size.

11. The method of treatment according to claim 10, wherein the embedded stem cells comprise an embedded stem cell diameter, and
wherein the maximal pore size is less than the embedded stem cell diameter, the maximal pore size is less than other cell diameters, and the maximal pore size is greater than embedded paracrine factor, cell product, nutrient, or waste diameters.

12. The method of treatment according to claim 8, wherein the nanofiber fabric comprises a biodegradable polymer.

13. The method of treatment according to claim 11, wherein the nanofiber fabric comprises at least one synthetic biocompatible material, a biological material, and any combination thereof.

14. The method of treatment according to claim 12, wherein the nanofiber fabric comprises at least one of poly(L-lactic acid), poly(lactic-co-glycolic acid) copolymer, polycaprolactone, poly(ethylene oxide), poly(ethylene terephthalate), poly(vinyl alcohol), collagen, and any combination thereof.

15. The method of treatment according to claim 8, wherein applying the therapeutic patch to an object comprises applying the therapeutic patch to at least one of a tissue, an organ or portion of an organ, a blood vessel, a muscle, a bone, a joint, a bypass graft, a medical device, and any combination thereof.

16. The method of treatment according to claim 15, wherein applying the therapeutic patch to an object comprises applying the therapeutic patch to a stent, a pacemaker, an implantable cardioverter-defibrillator, a pacemaker lead, an implantable cardioverter-defibrillator lead, a biventricular implantable cardioverter-defibrillator lead, an artificial heart, an artificial valve, a ventricular assist device, a balloon pump, a catheter, a central venous line, an orthopedic implant, wound healing packing, and wound healing gauze.

17. The method of treatment according to claim 8, wherein preparing a therapeutic patch comprises preparing a single-use therapeutic patch including stem cell-derived paracrine factors.

18. The method of treatment according to claim 17, wherein preparing the single-use therapeutic patch comprises at least one of directly absorbing conditioned media into the therapeutic patch, immersing the therapeutic patch in a homogenized cell solution, and lysing stem cells in the therapeutic patch.

19. The method of treatment according to claim 8, wherein preparing a therapeutic patch comprises preparing a reusable therapeutic patch including stem cells.

20. The method of treatment according to claim 19, wherein preparing the reusable therapeutic patch comprises:
seeding stem cells into the therapeutic patch; and
incubating the stem cells in the therapeutic patch.
